(19) United States
(12) Patent Application Publication
(10) Pub. No.: US 2026/0130589 A1
(43) Pub. Date: May 14, 2026

(54) PORTABLE OPHTHALMIC DIAGNOSTIC SYSTEM WITH ARTIFICIAL INTELLIGENCE PROCESSING

(71) Applicant: TeleMedC Group, Inc., Longmont, CO (US)

(72) Inventors: Para K. Segaram, Fernley, NV (US); Di Xiao, Brisbane, QLD (AU); Teck Jack Tan, Brisbane, QLD (AU); Vinayak Swarnkar, Chennai (IN); Yak Sheng Richard Lee, Singapore (SG)

(73) Assignee: TeleMedC Group, Inc., Longmont, CO (US)

(21) Appl. No.: 19/387,975

(22) Filed: Nov. 13, 2025

Related U.S. Application Data
(60) Provisional application No. 63/833,275, filed on Nov. 13, 2024.

(51) Int. Cl. A61B 3/18; A61B 3/00; A61B 3/11; A61B 3/117; A61B 3/14

(52) U.S. Cl. CPC A61B 3/185; A61B 3/0025; A61B 3/0058; A61B 3/112; A61B 3/1176; A61B 3/145

(57) ABSTRACT

A portable ophthalmic diagnostic system comprises a housing containing a red reflex illumination subsystem for visualizing lens and optical clarity, and a digital slit lamp subsystem with an adjustable dual-sided illumination source and digital imaging sensor for capturing eye images. The system includes an artificial intelligence processing interface that analyzes red reflex and slit-lamp image data to detect and classify ocular conditions including cataracts and optic nerve abnormalities. A colorimetric pupillary light reflex testing module assesses pupil constriction response using multicolor illumination. The system features a user interface displaying real-time images, diagnostic outputs, and AI-based classification results. The system enables computer-implemented methods for detecting ocular conditions through image capture, AI processing, red reflex analysis, cataract classification, pupillary response testing, and diagnostic report generation. The portable design includes telemedicine functionality for remote clinical consultation and cloud-based data storage for tracking disease progression.

### CROSS-REFERENCE TO RELATED APPLICATIONS

The present application relates to and claims the benefit of priority to U.S. Provisional Patent Application no. 63/833275 filed 13 Nov. 2024 which is hereby incorporated by reference in its entirety for all purposes as if fully set forth herein.

### BACKGROUND

### Field of the Invention

The disclosed invention relates to medical diagnostic devices, and more particularly to an AI-powered digital slit lamp system for eye examination that combines red reflex imaging, digital slit lamp technology, and colorimetric pupillary light reflex testing for detection and classification of eye conditions including cataracts and optic nerve diseases.

### Relevant Background

In the prior art, ophthalmic diagnostic equipment typically consists of separate specialized instruments for different examination types. Traditional slit lamps are stationary devices found in clinical settings that provide illumination and magnification for examining the anterior segment of the eye but often lack integrated digital imaging capabilities. These conventional systems require significant training to operate effectively and are not designed for portable use in non-clinical environments.

Pupillary light reflex testing has conventionally been performed using simple penlight examinations or specialized pupillometers that typically employ single-wavelength light sources, limiting the assessment of wavelength-specific responses. While some systems have attempted to analyze pupillary reflexes using digital imaging, they generally lack integration with other diagnostic modalities and comprehensive analysis capabilities. The separation of these diagnostic functions necessitates multiple devices and examinations, creating inefficiencies in clinical workflows.

Recent developments have attempted to address portability issues in ophthalmic diagnostics, with some systems incorporating smartphone technology for basic eye examinations. However, these solutions typically offer limited diagnostic capabilities compared to clinical equipment and lack the comprehensive integration of multiple examination modalities. Additionally, while some portable slit lamp designs exist, they often compromise on image quality or examination capabilities compared to their stationary counterparts.

The integration of artificial intelligence into ophthalmic diagnostics remains limited in commercially available systems. Traditional diagnostic approaches rely heavily on clinician interpretation of images rather than automated analysis, which introduces variability in diagnosis and requires substantial expertise. Furthermore, existing systems typically lack the ability to perform comprehensive examinations including both red reflex assessment and digital slit lamp imaging in a single portable device, particularly for cataract screening and optic nerve evaluation.

The technical challenge lies in developing an integrated portable system that combines multiple ophthalmic diagnostic capabilities including red reflex illumination, digital slit lamp functionality, and artificial intelligence-based analysis in a single device while maintaining clinical-grade diagnostic accuracy. Such a system would need to overcome limitations in portability, integration, and automated analysis to enable comprehensive eye examinations in diverse settings, particularly where access to traditional ophthalmic equipment is limited.

These and other deficiencies of the prior art are addressed by one or more embodiments of the disclosed invention. Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out hereafter.

The features and advantages described in this disclosure and in the following detailed description are not all-inclusive. Many additional features and advantages will be apparent to one of ordinary skill in the relevant art in view of the drawings, specification, and claims hereof. Moreover, it should be noted that the language used in the specification has been principally selected for readability and instructional purposes and may not have been selected to delineate or circumscribe the inventive subject matter; reference to the claims is necessary to determine such inventive subject matter.

### BRIEF DESCRIPTION OF THE DRAWINGS

Features and objects of the present invention and the manner of attaining them will become more apparent, and the invention itself will be best understood, by reference to the following description of one or more embodiments taken in conjunction with the accompanying drawings and figures imbedded in the text below and attached following this description.

FIG. 1 is an external perspective view of the portable ophthalmic diagnostic system showing the housing with integrated components, according to one embodiment of the present invention.

FIG. 2 is an external perspective view of the portable ophthalmic diagnostic system showing the touchscreen user interface display, according to one embodiment of the present invention.

FIG. 3A is a front view of the examination aperture and dual-sided illumination sources of the portable ophthalmic diagnostic system, according to one embodiment of the present invention

FIG. 3B is a front view of the examination aperture and dual-sided illumination sources of the portable ophthalmic diagnostic system, according to another embodiment of the present invention.

FIG. 4A is a cut-away drawing of the portable ophthalmic diagnostic system, according to one embodiment of the present invention.

FIG. 4B is a front cut-away drawing of ocular mechanisms of the portable ophthalmic diagnostic system, according to one embodiment of the present invention.

FIG. 4C is a top view of the ocular mechanisms of the portable ophthalmic diagnostic system, according to one embodiment of the present invention.

FIGS. 5A and 5B present a perspective and exploded view of a binocular version of the portable ophthalmic diagnostic system, according to one embodiment of the present invention.

FIG. 6 is a high level system diagram of the portable ophthalmic diagnostic system, according to one embodiment of the present invention.

FIG. 7 is a flowchart for one methodology for detecting and classifying ocular conditions according to one embodiment of the present invention.

FIG. 8 depicts a high-level block diagram of a machine capable of executing instructions and disclosed elements, according to one embodiment of the present invention.

The Figures depict embodiments of the present invention for purposes of illustration only. One skilled in the art will readily recognize from the following discussion that alternative embodiments of the structures and methods illustrated herein may be employed without departing from the principles of the invention described herein.

### SUMMARY OF THE INVENTION

The present disclosure relates to a portable ophthalmic diagnostic system that integrates red reflex imaging with digital slit lamp technology to provide comprehensive eye examination capabilities in a single handheld device. The system addresses the limitations of traditional ophthalmic examination tools by combining multiple diagnostic functions, enabling both anterior and posterior segment examination, and incorporating artificial intelligence for enhanced diagnostic accuracy.

In one aspect, the portable ophthalmic diagnostic system comprises a housing containing a red reflex illumination subsystem configured to generate reflected retinal illumination for visualizing lens and optical clarity. The red reflex subsystem may include a light-emitting diode (LED) array specifically optimized for retinal reflection analysis, providing superior visualization of optical clarity for early detection of cataracts and other lens opacities.

In one aspect, the system includes a digital slit lamp subsystem comprising an adjustable dual-sided illumination source positioned at approximately 45 degrees relative to an examination axis, and a digital imaging sensor configured to capture anterior and posterior eye images. The imaging sensor may comprise a digital camera with at least 1-megapixel resolution and optical magnification between 6× and 40×, enabling high-resolution, real-time imaging for detailed examination of the cornea, iris, lens, and retina.

In another aspect, the system incorporates an artificial intelligence (AI) processing unit communicatively coupled to the digital imaging sensor, the AI processing unit configured to analyze red reflex and slit-lamp image data to detect and classify ocular conditions including cataracts and optic nerve abnormalities. The AI processing unit may comprise a convolutional neural network trained on ophthalmic image datasets to perform image segmentation and pattern recognition, reducing human error and enhancing diagnostic efficiency through AI-powered analysis.

The system of the present invention includes a colorimetric pupillary light reflex (cPLR) testing module configured to assess pupil constriction response using multicolor illumination. The cPLR module may employ multi-wavelength light sources to evaluate color-specific pupillary responses, providing additional diagnostic information for detection and classification of eye conditions.

In one aspect, the system features a user interface configured to display real-time images, diagnostic outputs, and AI-based classification results. The user interface may comprise a touchscreen display configured to show live imaging feeds and diagnostic overlays, streamlining the diagnostic approach and aiding in early disease detection.

According to another aspect of the present disclosure, a computer-implemented method for detecting and classifying ocular conditions is provided. The method includes capturing red reflex and slit-lamp images of an eye using a handheld ophthalmic device, processing the captured images through an artificial intelligence algorithm trained on ophthalmic image data, analyzing the color, intensity, and symmetry of the red reflex to identify lens opacity or cataract presence, classifying the detected cataract as one of nuclear, cortical, or posterior based on image pattern recognition, performing a colorimetric pupillary light reflex test to measure pupillary response under multiple color wavelengths, and generating a diagnostic report including image data, AI-derived assessment, and a classification of ocular condition severity.

The system may include cloud-based data storage and connectivity features that store examination data and allow for remote consultation, integration with patient records, and telemedicine capabilities. This makes the system particularly valuable in telemedicine applications, where it can capture and upload images to a cloud-based platform for remote analysis by clinical specialists, improving accessibility to comprehensive eye examinations, particularly in resource-constrained environments.

Although illustrative embodiments of one or more aspects are provided herein, the disclosed processes may be implemented using any number of techniques. The disclosure is not limited to the illustrative or specific embodiments, any drawings, and any techniques illustrated herein, including any exemplary designs and embodiments illustrated and described herein, and may be modified within the scope of the appended claims along with their full scope of equivalents.

### DESCRIPTION

The disclosed invention includes devices, systems, and methods comprising a portable slit lamp system for eye examination that combines red reflex imaging, digital slit lamp technology, and colorimetric pupillary light reflex testing for detection and classification of eye conditions including, among other things, cataracts and optic nerve diseases. The present disclosure relates to a handheld ophthalmic diagnostic device that integrates red reflex imaging with a digital slit lamp, addressing the limitations of traditional ophthalmic examination tools. Traditional slit lamps and red reflex technology often lack integration, creating inefficient workflows and limiting comprehensive eye analysis in a single sitting. While traditional slit lamps are effective for anterior segment examination, they have limitations in examining the posterior segment of the eye. Additionally, separate diagnostic tools for different eye examinations pose a significant barrier to timely and accurate diagnosis, especially in rural and remote areas where resource constraints and logistical challenges leave many eye conditions undiagnosed until they progress to an advanced stage.

To make the objectives, technical solutions, and advantages of this application clearer, the following further describes this application in detail with reference to the accompanying drawings. However, example implementations can be implemented in a plurality of forms and should not be construed as being limited to the implementations described herein. Identical reference numerals in the accompanying drawings denote identical or similar structures. Therefore, repeated description thereof is omitted. Expressions of positions and directions in embodiments of this application are described by using the accompanying drawings as an example. However, changes may be also made as required, and all the changes fall within the protection scope of this application. The accompanying drawings in embodiments of this application are merely used to illustrate a relative position relationship and do not represent an actual scale.

It should be noted that specific details are set forth in the following descriptions for ease of understanding this application. However, this application can be implemented in a plurality of manners different from those described herein, and a person skilled in the art can perform similar promotion without departing from the connotation of this application. Therefore, this application is not limited to the following disclosed specific implementations.

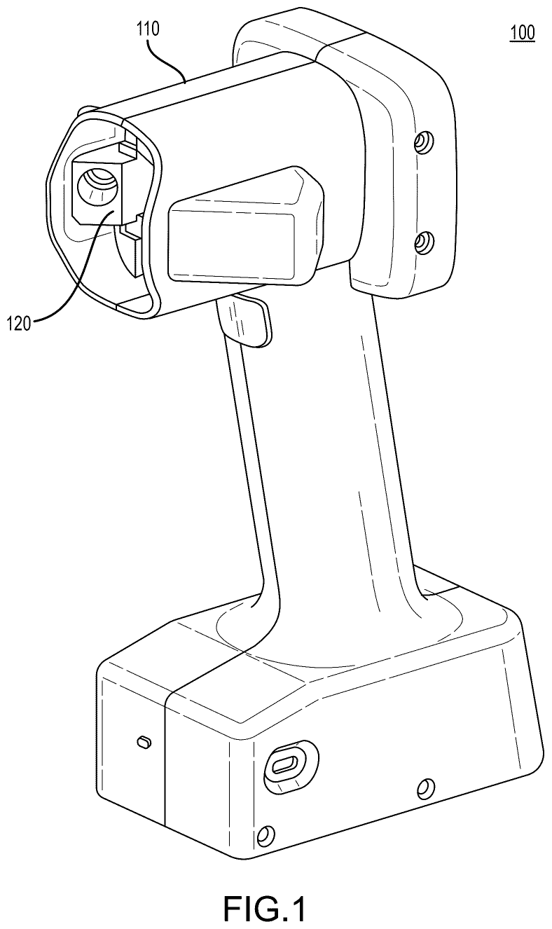

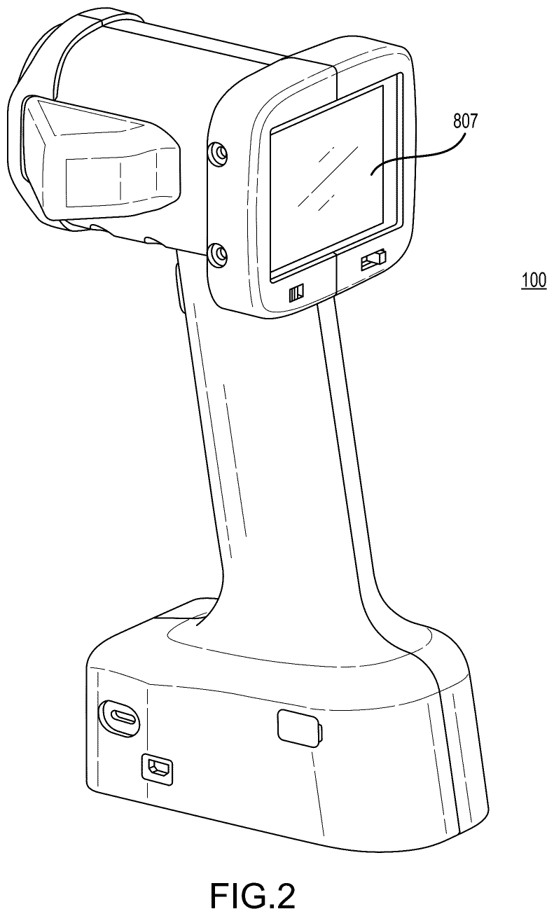

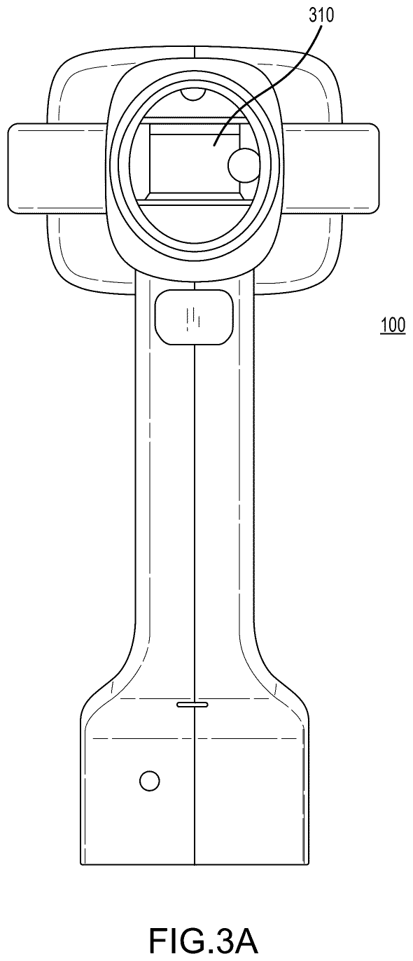

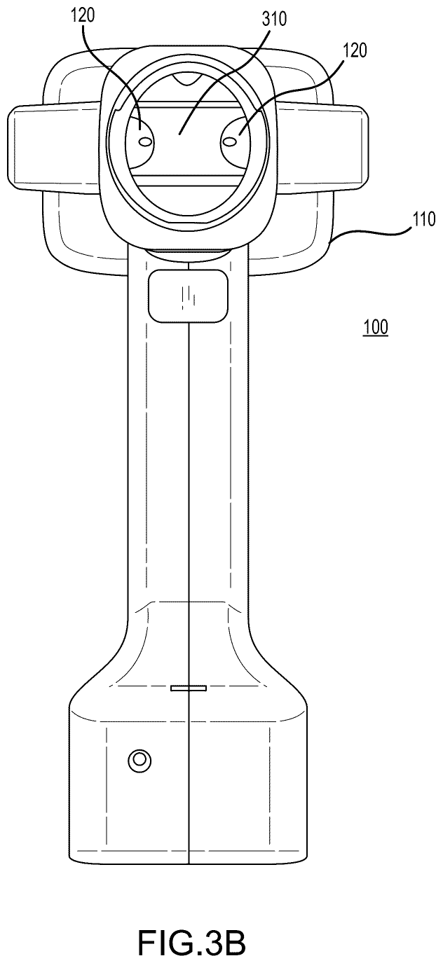

Referring to FIGS. 1-4, a portable monocular ophthalmic diagnostic system 100 includes a housing 110 that contains various subsystems for ophthalmic examination. The portable ophthalmic diagnostic system is designed for comprehensive eye examination and diagnosis in a compact, mobile form factor that enables use in various clinical settings.

The housing contains a red reflex illumination subsystem 30 configured to generate reflected retinal illumination for visualizing lens and optical clarity. The red reflex subsystem 310 comprises a light-emitting diode (LED) array optimized for retinal reflection analysis. The LED array includes multiple light sources arranged in a specific pattern to provide uniform illumination of the retina, enabling clear visualization of any opacities or abnormalities in the eye's optical path.

The portable ophthalmic diagnostic system further includes a digital slit lamp subsystem 120 comprising an adjustable dual-sided illumination source positioned at approximately 45 degrees relative to an examination axis. This this specific angular positioning allows for optimal illumination of the eye structures during examination, creating the necessary contrast for detailed visualization of ocular tissues. The digital slit lamp subsystem 120 includes a digital imaging sensor configured to capture anterior and posterior eye images. The digital imaging sensor 430 is strategically positioned relative to the illumination sources 420 to capture optimal images during examination.

The digital imaging sensor 430 includes a digital camera with at least 1-megapixel resolution and optical magnification between 6× and 40×. This high resolution and variable magnification capability enables detailed examination of fine ocular structures, from the corneal epithelium to the retinal surface. The magnification can be adjusted based on the specific examination requirements, allowing for both wide-field views and detailed microscopic examination of eye tissues.

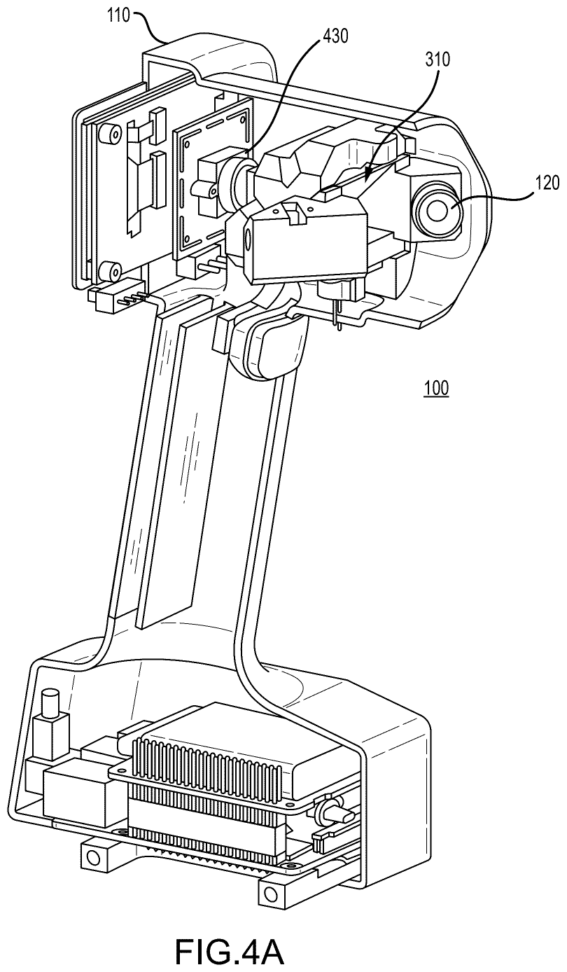

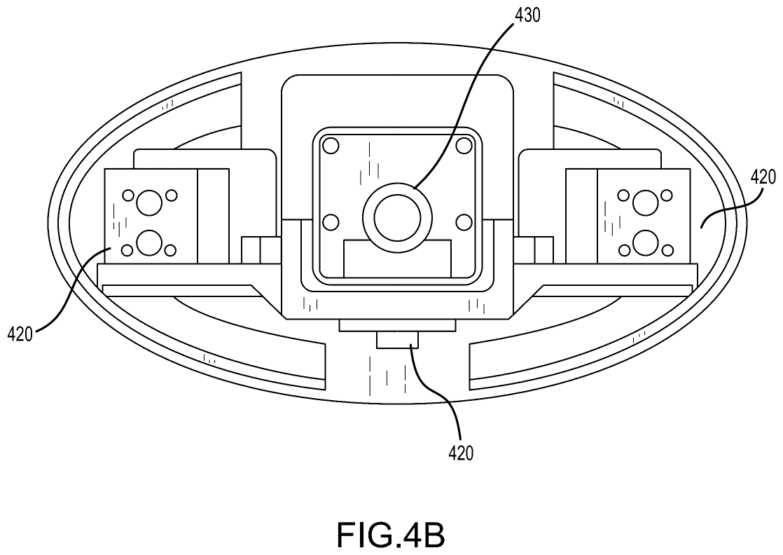

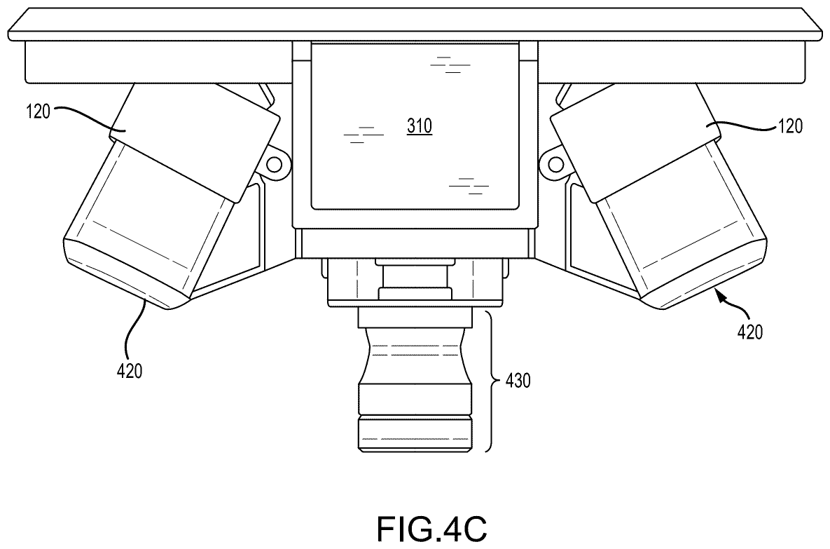

The internal arrangement of components including the illumination subsystems, digital imaging sensor, and processing hardware, as shown in FIGS. 4A, 4B, and 4C enables the device to perform all required functions in a portable form factor. The digital slit lamp subsystem 120 works in conjunction with the red reflex illumination subsystem 310 to provide comprehensive imaging capabilities necessary for accurate diagnosis and classification of ocular conditions.

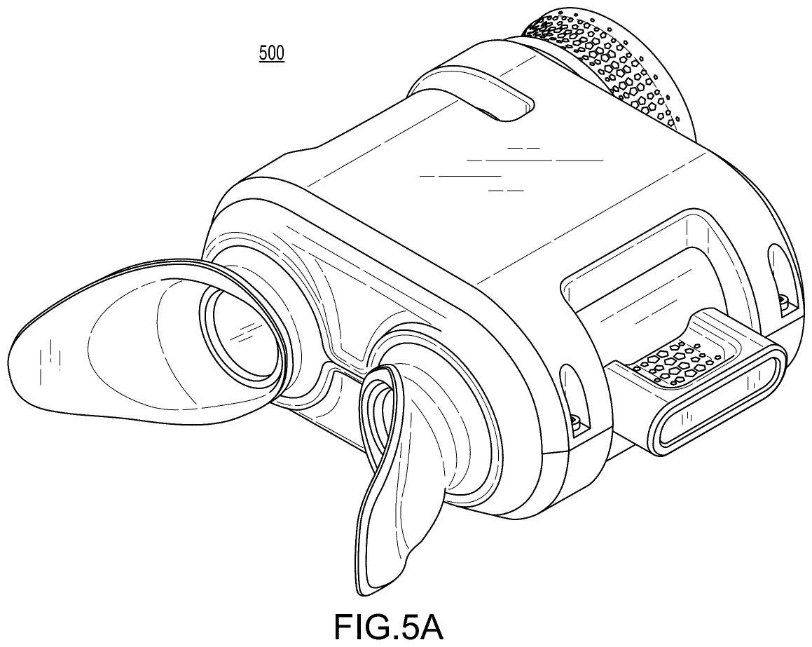

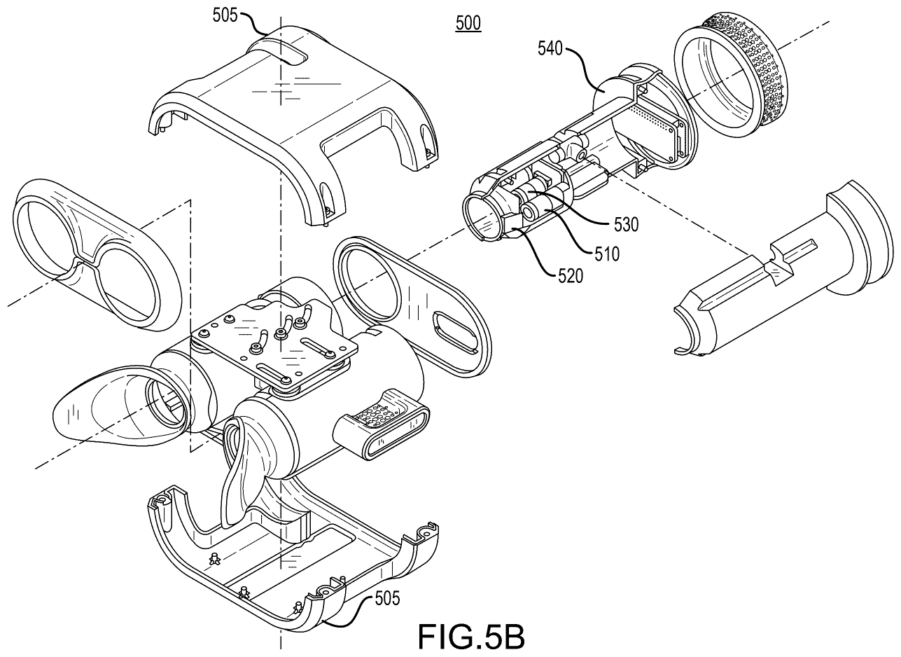

In another embodiment of the present invention, and with additional reference to FIGS. 5A and 5B, the portable ophthalmic diagnostic system is implemented in a binocular configuration 500 to provide enhanced stability, ergonomic balance, and examination efficiency. Operator steadiness and patient alignment are further optimized through the binocular housing layout. The binocular design therefore replaces the single optical channel with a symmetrical dual-aperture assembly that aligns naturally with both eyes of the operator or patient, providing a balanced handheld form factor that minimizes lateral drift during prolonged scans. In one embodiment each optical channel comprises a precision-matched optical lens stack 510, a dual-sided illumination source 520 positioned at approximately 45 degrees relative to the examination axis, and a dedicated imaging sensor 530 configured to capture high-resolution anterior and posterior eye images. Both channels are optically synchronized through a unified control module to ensure consistent magnification, illumination intensity, and focal alignment across the left-and right-eye pathways.

The binocular arrangement enables the examiner to maintain a stable stance during bilateral eye assessments, reducing repositioning time and improving workflow continuity. The outer housing 505 incorporates a central electronics chassis 540 that remains stationary while the optical enclosure rotates approximately 180 degrees about the vertical axis. This rotational capability allows the examiner to switch between left-and right-eye scanning modes simply by flipping the enclosure, eliminating the need for cable reorientation or device recalibration. The electronic chassis hosts the primary imaging processors, connectively to the artificial-intelligence (AI) computing module, and all associated power and communication circuits in a modular configuration that permits external access for assembly, maintenance, and component testing. This modular design ensures serviceability and allows for field upgrades or replacement of optical or processing elements without compromising the sealed optical path or patient-facing surfaces.

Ergonomic refinements such as grip contouring, inter-ocular spacing, and weight distribution have been optimized for both clinician and patient comfort. The use of lightweight, high-strength polymers and internal honeycomb structures reduces device mass while maintaining rigidity and vibration resistance.

In this embodiment, the secondary eye cup—the eye interface opposite the primary imaging channel—serves multiple integrated functions. In one embodiment, it houses a rechargeable battery module, an illumination driver board, and a patient fixation light source used to provide a constant visual target for the non-imaged eye during scanning. The fixation light maintains equivalent luminance between both visual fields so that pupil dilation remains symmetrical, an essential condition for consistent red-reflex and colorimetric pupillary light reflex (cPLR) measurements. The binocular configuration therefore mitigates the common variability observed in monocular testing, where asymmetric illumination can cause differential pupillary constriction and inconsistent readings. During operation, the fixation light intensity and wavelength are automatically coordinated with the active scanning light of the imaged eye, ensuring that both pupils remain at comparable saturation levels and optimizing the accuracy of the cPLR and AI-derived pupil-response analysis.

The binocular system preserves all diagnostic modalities of the monocular device while extending them to dual-eye operation. An optical channel contains dual 45-degree slit-lamp illumination sources coupled to a high-resolution digital imaging sensor. The imaging sensor operates in to capture either independent left-and right-eye datasets of a single eye for enhanced depth analysis. In another embodiment, when system operates in stereo mode, the captured image pairs are processed by the AI module to analyze the corneal or lenticular surface, improving the device's capacity to detect surface irregularities, cataract morphology, and anterior-segment asymmetry. The AI processing unit, as previously described, comprises a convolutional neural network trained on ophthalmic image datasets; in the binocular system, this network is further optimized to analyze cross-eye symmetry by comparing corresponding anatomical landmarks between the two image sets. This feature enables quantitative assessment of bilateral conditions such as anisocoria, asymmetric cataract development, and optic-nerve disease progression.

In this embodiment, the slit generation module (SDM) 510 and the mirror 520 form the slit lamp component of one side of the device. A splitter cube plus the surrounding LED light source and infrared light is consistent with the monocular version of the invention. In a preferred embodiment data with respect to one eye is captured at a time. The system is then rotated to use the same image capturing components to capture data with respect to the other eye. As compared to the monocular device, the slit lamp components are placed in parallel on two sides with the optical axis that then +/−45 degrees in the monocular device. The binocular system maintains the use of two +/− mirrors 520 to reflect the slit light to form the same +/−45 degree light projections on the corneal surface.

The mechanical interface of the binocular housing has been engineered for operator comfort and long-term durability. The dual-eyepiece configuration distributes contact pressure evenly across both eyes and facial surfaces, thereby reducing localized strain during extended screening sessions. The enclosure incorporates soft medical-grade elastomer seals for patient contact points and removable alignment gaskets for hygienic replacement. Cooling channels integrated into the internal structure maintain thermal stability of both imaging sensors and LED illumination arrays. The outer shell geometry allows one-handed operation while preserving balanced center-of-gravity alignment directly above the clinician's grip point, an ergonomic improvement validated during human-factors testing.

The binocular configuration also improves data acquisition efficiency. During a typical examination session, both eyes can be scanned sequentially without changing device orientation or recalibrating optical parameters. The device's embedded software recognizes the rotational position of the enclosure and automatically switches between left-and right-eye imaging profiles. This automated reconfiguration adjusts camera parameters, illumination balance, and AI analysis templates, accordingly, thereby reducing setup time and ensuring consistent imaging across sessions. All acquired data—including red-reflex images, slit-lamp photographs, and cPLR response curves—are timestamped and stored in the on-board memory or transmitted through the cloud-based telemedicine interface for remote review.

From an electronic standpoint, the binocular version incorporates identical imaging modules for each channel but employs a shared connectivity to the AI processing core to optimize real-time performance. The AI unit executes dual parallel processing threads that analyze image data concurrently, with results displayed on the integrated touchscreen interface in the form of diagnostic overlays, colorimetric heat maps, and AI-based classification outputs. The power management system monitors thermal and energy profiles from both channels and redistributes processing load as needed to maintain optimal performance while conserving battery life.

In practical use, the binocular embodiment greatly enhances diagnostic reliability. The combination of mechanical symmetry, balanced illumination, and dual-sensor imaging produces higher-quality, noise-resistant data that improve AI accuracy for cataract classification and optic-nerve anomaly detection. When integrated with telemedicine workflows, the binocular device enables paired-eye comparisons in a single session, allowing remote specialists to assess asymmetry in optic-disc appearance, retinal reflection uniformity, or differential cataract opacity with high precision.

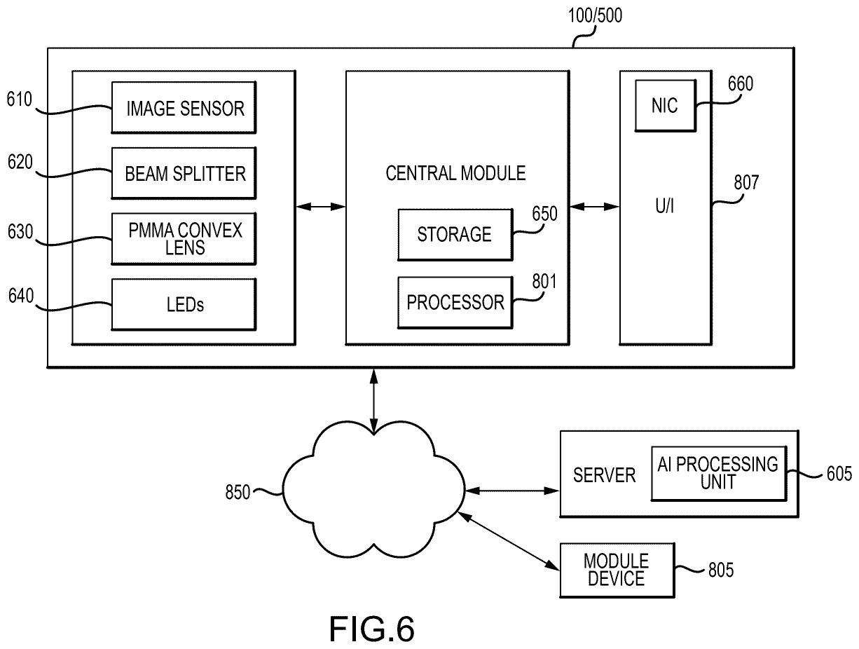

As illustrated in FIG. 6, the portable ophthalmic diagnostic system includes an artificial intelligence (AI) processing unit 605 communicatively coupled to the digital imaging sensor 610. The AI processing unit is configured to analyze red reflex and slit-lamp image data collected through the beam splitter, 620, the lens module 630, and LEDS 640 to detect and classify ocular conditions including cataracts and optic nerve abnormalities. The AI processing unit comprises a convolutional neural network trained on ophthalmic image datasets to perform image segmentation and pattern recognition. This neural network architecture enables automated identification of pathological features in ocular images, providing decision support for clinicians during diagnosis.

The AI processing unit compares current examination data with prior patient data to track disease progression. This longitudinal analysis capability allows clinicians to monitor changes in ocular conditions over time, facilitating early intervention and treatment adjustment based on disease progression patterns.

As discussed herein, the handheld ophthalmic diagnostic device includes a red reflex illumination system comprising a specialized light source designed to generate red reflex by reflecting off the retina, highlighting opacities in the eye's optical system. The device enables both anterior and posterior segment examination in one unit, allowing for assessment of a wider range of eye diseases, with the red reflex component offering superior visualization of optical clarity for early detection of cataracts and other lens opacities.

The device's incorporation of AI software and a unified interface allows real-time image processing, enhancement, and analysis of the red reflex data along with slit-lamp observations. The integration of cloud-based advanced AI-powered diagnostic algorithms assists clinicians in rapidly and accurately identifying abnormalities, reducing human error and enhancing diagnostic efficiency.

The device provides real-time feedback through an AI system that offers the clinician real-time diagnostic suggestions, flags potential issues, and generates reports. This expands access to expert care and makes the device a practical solution for use in pediatric wards, maternity wards, and remote areas where healthcare resources and specialists are limited.

The device further includes data storage 650 and connectivity features 660 that store examination data and allow for remote consultation, integration with patient records, and telemedicine capabilities. This makes the device particularly valuable in telemedicine, where it can capture and upload images to a cloud-based platform for remote analysis by clinical specialists. This connectivity feature enables secure storage of patient data and facilitates access to historical examination records for comparative analysis. The cloud-based architecture ensures that patient data remains accessible across different clinical settings while maintaining data security and patient privacy.

The device disclosed is portable, battery-powered, and includes a docking station for easy charging, enhancing its usability in various healthcare settings, particularly in resource-constrained environments.

Through the adoption of modular electronics, and bilateral imaging synchronization, the binocular version of the portable ophthalmic diagnostic system represents a substantial advancement over the initial monocular design. It maintains the core innovations of the invention—integrated red-reflex imaging, digital slit-lamp microscopy, and AI-assisted analysis—while expanding diagnostic scope, stability, and usability. The result is a clinically robust, portable binocular ophthalmic platform that unifies optical, electronic, and computational systems into a single, ergonomic instrument capable of comprehensive bilateral ocular evaluation across diverse clinical and telemedicine environments.

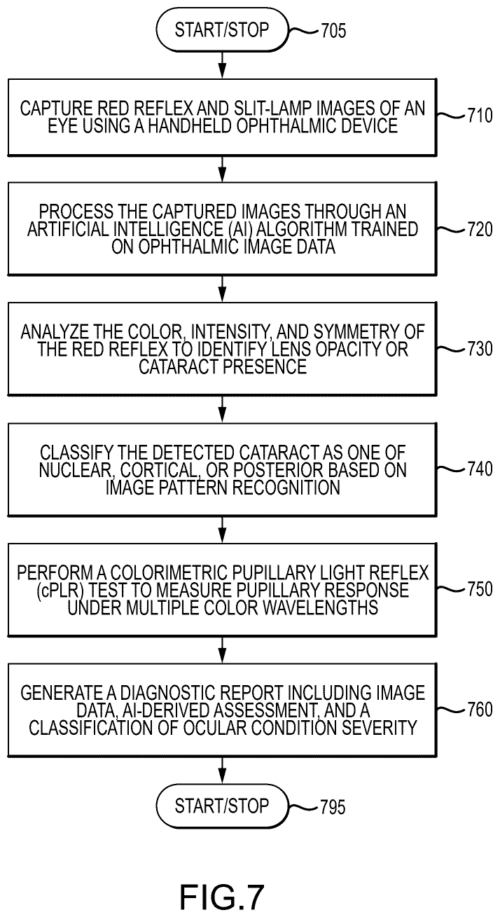

In another embodiment of the present invention and with additional reference to FIG. 7, a computer-implemented method for detecting and classifying ocular conditions includes the following steps.

The process begins 705 with capturing 710 red reflex and slit-lamp images of an eye using a handheld ophthalmic device. The handheld ophthalmic device includes a housing with integrated red reflex illumination subsystem and digital imaging components. The device synchronizes illumination intensity with image capture to ensure optimal exposure. Ophthalmic lenses are arranged with strategically positioned illumination sources to capture both red reflex and slit-lamp images during a single examination session.

Processing 720 the captured images through an artificial intelligence (AI) algorithm trained on ophthalmic image data housed, in one embodiment, on a server is the next step in the process. The AI algorithm employs image segmentation to delineate ocular structures including the cornea, iris, and lens. The algorithm identifies abnormalities based on deviations in red reflex symmetry and luminance, comparing the captured images to a reference database of normal and pathological eye images.

The color, intensity, and symmetry of the red reflex is analyzed to identify lens opacity or cataract presence. The AI algorithm evaluates 730 the captured red reflex images using multiple parameters. In addition, the system utilizes the dual-sided illumination sources positioned at approximately 45 degrees relative to the examination axis to ensure consistent slit illumination for accurate analysis of characteristics.

Classifying 740 a detected cataract as one of nuclear, cortical, or posterior based on image pattern recognition is the next step in the disclosed process. The AI algorithm, following the procedural steps outlined in FIG. 6, applies pattern recognition techniques to the captured images to determine the specific type of cataract present. The system generates a quantitative opacity score indicating cataract severity based on the analysis of the red reflex and slit-lamp images.

Additionally, a colorimetric pupillary light reflex (cPLR) test is performed 750 to measure pupillary response under multiple color wavelengths. The test includes measuring response times to red, green, and blue light stimuli. As discussed herein, the device includes LED arrays capable of producing specific color wavelengths for the cPLR test. The system records pupillary responses to each color stimulus and incorporates this data into the overall assessment.

Lastly, the present invention generates 760 a diagnostic report including image data, AI-derived assessment, and a classification of ocular condition severity. Following the operational sequence, the system compiles examination results into a comprehensive diagnostic report ending the process 795. The report includes the quantitative opacity score, cataract classification, and pupillary response data.

The method further includes displaying real-time AI feedback on the device interface to guide examiner positioning and focus using the touchscreen display. This feature ensures proper alignment of the device during image capture, improving the quality and consistency of the captured images.

In a preferred embodiment, the method further comprises storing examination data in a cloud server accessible via secure authentication allowing healthcare providers to access patient records remotely and track changes in ocular conditions over time.

Lastly, the method further comprises transmitting examination data for remote clinical validation through a telemedicine network. After image capture and AI analysis, the data can be transmitted to specialists for review and validation, enabling access to expert opinions even in remote or underserved areas.

Compared with prior art, the present invention provides a RedSlit medical device integrating a digital slit lamp with red reflex imaging and AI, and has the following beneficial effects:
Enables rapid, comprehensive eye assessments by combining advanced red reflex imaging, slit-lamp analysis, and colorimetric pupillary light reflex (cPLR) testing within a single, portable device. This integration allows for efficient screening and early detection of ocular conditions in both clinical and community-based settings.
Facilitates precise differentiation between retinal and optic nerve pathologies, guiding further evaluation and treatment. The combination of red reflex imaging, slit-lamp microscopy, and cPLR testing provides crucial insights into the underlying cause of vision loss, enabling more accurate diagnosis and targeted interventions.
Enhances the accuracy and efficiency of cataract classification and grading, aligning with World Health Organization (WHO) standards. The system's ability to automatically classify cataract types (nuclear, cortical, and posterior subcapsular) ensures consistent diagnosis and facilitates timely treatment planning.
Leverages advanced artificial intelligence (AI) and machine learning algorithms to analyze complex ophthalmic data, improving the overall accuracy and efficiency of eye examinations. This enables more reliable diagnostic decisions and reduces the risk of missed diagnoses or errors in ocular assessment.
Promotes portable and systemized integration of cutting-edge imaging techniques and AI-driven analysis, allowing for comprehensive eye evaluations in various clinical and community-based settings. This integration addresses the limitations of traditional slit lamp microscopy and expands the reach of advanced ophthalmic diagnostics to resource-limited environments.

The invention combines advanced red reflex imaging, digital slit lamp microscopy, and artificial intelligence (AI) to create a portable, systemized medical device called RedSlit.

The RedSlit device comprises a custom-designed slit lamp system integrated with a red reflex imaging module and a colorimetric pupillary light reflex (cPLR) testing unit. The slit lamp system utilizes dual 45-degree illumination angles to capture high-resolution images of anterior eye segments generating nuclear imaging. The red reflex imaging module projects bright light through the eye to visualize the reflection from the retina, while the cPLR testing unit uses colored light stimuli to activate distinct photoreceptor pathways.

The device is designed for rapid, comprehensive eye assessments, enabling efficient screening and early detection of ocular conditions in both clinical and community-based settings. It incorporates advanced AI algorithms and machine learning models to analyze the collected data, including red-reflex images, slit-lamp photographs, and cPLR results. These models are trained on large-scale datasets of various ocular abnormalities, including cataracts, retinal degeneration, and optic nerve disorders.

It will be also understood that when an element is referred to as being “on,” “attached” to, “connected” to, “coupled” with, “contacting”, “mounted” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used to describe one element or feature's relationship to another element(s) or feature(s) as illustrated in the figures. Such spatially relative terms are intended to encompass different orientations of a device in use or operation in addition to the orientation depicted in the figures. For example, if a device in the figures is inverted, elements described as “under,” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

Included in the description are flowcharts depicting examples of the methodology which may be used in a travel system for individuals with cognitive disabilities. In the following description, it will be understood that each block of the flowchart illustrations, and combinations of blocks in the flowchart illustrations, can be implemented by computer program instructions. These computer program instructions may be loaded onto a computer or other programmable apparatus to produce a machine such that the instructions that execute on the computer or other programmable apparatus create means for implementing the functions specified in the flowchart block or blocks. These computer program instructions may also be stored in a computer-readable memory that can direct a computer or other programmable apparatus to function in a particular manner such that the instructions stored in the computer-readable memory produce an article of manufacture including instruction means that implement the function specified in the flowchart block or blocks. The computer program instructions may also be loaded onto a computer or other programmable apparatus to cause a series of operational steps to be performed in the computer or on the other programmable apparatus to produce a computer implemented process such that the instructions that execute on the computer or other programmable apparatus provide steps for implementing the functions specified in the flowchart block or blocks.

Accordingly, blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware-based computer systems that perform the specified functions or steps, or combinations of special purpose hardware and computer instructions.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

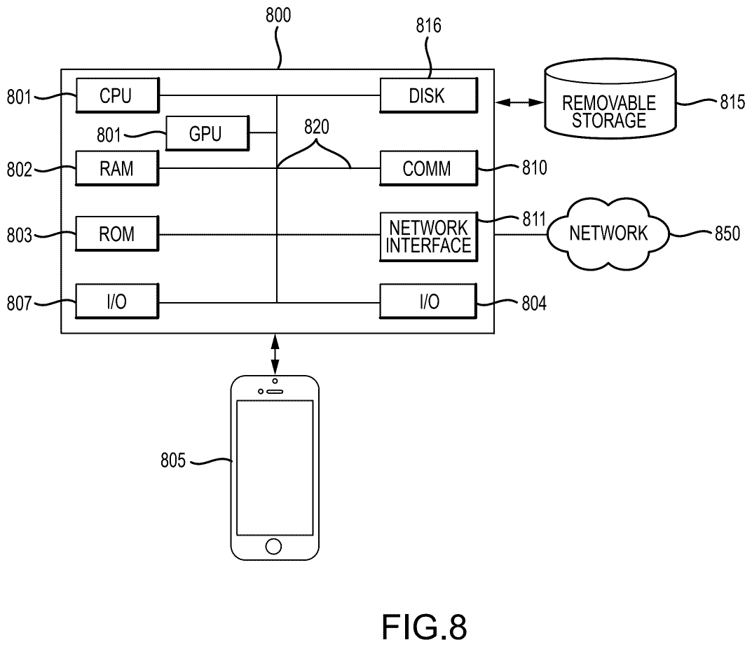

One of reasonable skill will also recognize that portions of the present invention may be implemented on a general-purpose mobile computing system, such as a smartphone, a personal communication device, a mobile device, a notebook computer, a tablet, or the like. FIG. 8 is a generalized block diagram of a computer system in which software-implemented processes of the present invention may be embodied. As shown, system 8 comprises a central processing unit(s) (CPU) or processor(s) 801 coupled to a random-access memory (RAM) 802, a graphics processor unit(s) (GPU) 820, a read-only memory (ROM) 803, a touchscreen or user interface 807, a display or video adapter 804 connected to a display device 805, a mass storage device 815 (e.g., flash memory, disk, or the like), a fixed (mass) storage device 816 (e.g., flash memory, a hard disk), a communication (COMM) port(s) or interface(s) 810, and a network interface card (NIC) or controller 811 (e.g., cellular, Ethernet, WIFI). Although not shown separately, various antennae and a real time system clock is included with the system 800, in a conventional manner.

CPU 801 comprises a suitable processor for implementing the present invention. The CPU 801 communicates with other components of the system via a bi-directional system bus 820 (including any necessary input/output (I/O) controller 807 circuitry and other “glue” logic). The bus, which includes address lines for addressing system memory, provides data transfer between and among the various components. Random-access memory 802 serves as the working memory for the CPU 801. The read-only memory (ROM) 803 contains the basic input/output system code (BIOS), a set of low-level routines in the ROM that application programs and the operating systems can use to interact with the hardware, including reading characters from the touchscreen or keyboard, outputting characters to screens or printers, and so forth.

Mass storage devices 815, 816 provide persistent storage on fixed and removable media, such as magnetic, optical, or magnetic-optical storage systems, flash memory, or any other available mass storage technology. The mass storage may be shared on a network 850, or it may be a dedicated mass storage. As shown in FIG. 8, fixed storage 816 stores a body of program and data for directing operation of the computer system, including an operating system, user application programs, driver, and other support files, as well as other data files of all sorts. Typically, the fixed storage 816 serves as the main memory for the system.

In basic operation, program logic (including that which implements methodology of the present invention described below) is loaded from the removable storage 815 or fixed storage 816 into the main (RAM) memory 802, for execution by the CPU 801. During operation of the program logic, the system 800 accepts user input from a keyboard and pointing device, as well as speech-based input from a voice recognition system (not shown). The user interface permits selection of application programs, entry of keyboard-based input or data, and selection and manipulation of individual data objects displayed on the screen or display device 805. Likewise, the pointing device, such as a mouse, track ball, pen device, touch screen, or the like, permits selection and manipulation of objects on the display device. In this manner, these input devices support manual user input for any process running on the system.

The computer system 800 displays text and/or graphic images and other data on the display device 805. The video adapter 804, which is interposed between the display 805 and the system's bus, drives the display device 805. The video adapter 804, which includes video memory accessible to the CPU 801, provides circuitry that converts pixel data stored in the video memory to a raster signal suitable for use by a monitor or touchscreen. A hard copy of the displayed information, or other information within the system 800, may be obtained from a printer or other output device.

The system itself communicates with other devices (e.g., other computers) via the network interface card (NIC) 811 connected to a network 850 (e.g., cellular network, Wi-Fi network, Bluetooth wireless network, or the like). The system 800 may also communicate with local occasionally connected devices (e.g., serial cable-linked devices) via the communication (COMM) interface 810, which may include a RS-232 serial port, a Universal Serial Bus (USB) interface, or the like. Devices that will be commonly connected locally to the interface 810 include laptop computers, handheld organizers, digital cameras, and the like.

The system itself communicates with other devices (e.g., other handheld devices or computers) via the NIC 811 connected to a network (e.g., cellular network, Wi-Fi network, Bluetooth wireless network, etc.). The system 800 may also communicate with local occasionally connected devices (e.g., serial cable-linked devices) via the COMM interface 810, which may include a RS-232 serial port, a Universal Serial Bus (USB) interface, or the like. Devices that will be commonly connected locally to the interface 810 include laptop computers, handheld computers, digital cameras, etc.

The system may be implemented through various wireless networks and their associated communication devices. Such networks may include modems, mainframe computers, or servers, such as a gateway computer or application server which may have access to a database. A gateway computer serves as a point of entry into each network and may be coupled to another network by means of a communications link. The gateway may also be directly or indirectly coupled to one or more devices using a communications link or may be coupled to a storage device such as a data repository or database.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve the manipulation of information elements. Typically, but not necessarily, such elements may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” “words,” “materials,” etc. These specific words, however, are merely convenient labels and are to be associated with appropriate information elements.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

Upon reading this disclosure, those of skill in the art will appreciate still additional alternative structural and functional designs for a system and a process for cognitive training through the disclosed principles herein. Thus, while embodiments and applications have been illustrated and described, it is to be understood that the disclosed embodiments are not limited to the precise construction and components disclosed herein. Various modifications, changes, and variations, which will be apparent to those skilled in the art, may be made in the arrangement, operation and details of the method and apparatus disclosed herein without departing from the spirit and scope of the invention.

It will also be understood by those familiar with the art, that the invention may be embodied in other specific forms without departing from the spirit or essential characteristics thereof. Likewise, the naming and division of the modules, managers, functions, systems, engines, layers, features, attributes, methodologies, and other aspects are not mandatory or significant, and the mechanisms that implement the invention or its features may have different names, divisions, and/or formats. Furthermore, as will be apparent to one of ordinary skill in the relevant art, the modules, managers, functions, systems, engines, layers, features, attributes, methodologies, and other aspects of the invention can be implemented as software, hardware, firmware, or any combination of the three. Of course, wherever a component of the present invention is implemented as software, the component can be implemented as a script, as a standalone program, as part of a larger program, as a plurality of separate scripts and/or programs, as a statically or dynamically linked library, as a kernel loadable module, as a device driver, and/or in every and any other way known now or in the future to those of skill in the art of computer programming. Additionally, the present invention is in no way limited to implementation in any specific programming language, or for any specific operating system or environment.

We claim:

1. A portable ophthalmic diagnostic system comprising:
a housing containing a red reflex illumination subsystem configured to generate reflected retinal illumination for visualizing lens and optical clarity;
a digital slit lamp subsystem comprising an adjustable dual-sided illumination source positioned at approximately 45 degrees relative to an examination axis, and a digital imaging sensor configured to capture anterior and posterior eye images;
an artificial intelligence (AI) processing unit communicatively coupled to the digital imaging sensor, the AI processing unit configured to analyze red reflex and slit-lamp image data to detect and classify ocular conditions including cataracts and optic nerve abnormalities;
a colorimetric pupillary light reflex (cPLR) testing module configured to assess pupil constriction response using multicolor illumination; and
a user interface configured to display real-time images, diagnostic outputs, and AI-based classification results.

2. The system of claim 1, wherein the red reflex subsystem comprises a light-emitting diode (LED) array optimized for retinal reflection analysis.

3. The system of claim 1, wherein the imaging sensor comprises a digital camera with at least 1-megapixel resolution and optical magnification between 6× and 40×.

4. The system of claim 1, wherein the AI processing unit comprises a convolutional neural network trained on ophthalmic image datasets to perform image segmentation and pattern recognition.

5. The system of claim 1, further comprising a cloud-based data storage module for transmitting and storing examination results.

6. The system of claim 1, wherein the user interface comprises a touchscreen display configured to show live imaging feeds and diagnostic overlays.

7. The system of claim 1, wherein the cPLR module employs multi-wavelength light sources to evaluate color-specific pupillary responses.

8. The system of claim 1, wherein the system further includes a rechargeable battery and docking station for portable operation.

9. The system of claim 1, wherein the AI processing unit compares current examination data with prior patient data to track disease progression.

10. The system of claim 1, wherein the device integrates telemedicine functionality to transmit captured images to a remote clinician for review.

11. A computer-implemented method for detecting and classifying ocular conditions, comprising:
capturing red reflex and slit-lamp images of an eye using a handheld ophthalmic device;
processing the captured images through an artificial intelligence (AI) algorithm trained on ophthalmic image data;
analyzing the color, intensity, and symmetry of the red reflex to identify lens opacity or cataract presence;
classifying the detected cataract as one of nuclear, cortical, or posterior based on image pattern recognition;
performing a colorimetric pupillary light reflex (cPLR) test to measure pupillary response under multiple color wavelengths; and
generating a diagnostic report including image data, AI-derived assessment, and a classification of ocular condition severity.

12. The method of claim 11, wherein the AI algorithm identifies abnormalities based on deviations in red reflex symmetry and luminance.

13. The method of claim 11, wherein the AI algorithm compares the captured images to a reference database of normal and pathological eye images.

14. The method of claim 11, further comprising storing examination data in a cloud server accessible via secure authentication.

15. The method of claim 11, further comprising generating a quantitative opacity score indicating cataract severity.

16. The method of claim 11, wherein the AI algorithm employs image segmentation to delineate ocular structures including the cornea, iris, and lens.

17. The method of claim 11, further comprising transmitting examination data for remote clinical validation through a telemedicine network.

18. The method of claim 11, wherein the colorimetric pupillary light reflex test includes measuring response times to red, green, and blue light stimuli.

19. The method of claim 11, wherein the handheld device synchronizes illumination intensity with image capture to ensure optimal exposure.

20. The method of claim 11, further comprising displaying real-time AI feedback on the device interface to guide examiner positioning and focus.
